(19) United States
(12) Patent Application Publication
(10) Pub. No.: US 2025/0248688 A1
(43) Pub. Date: Aug. 7, 2025

(54) SYSTEMS AND METHODS FOR ULTRASOUND ANGLE VISUALIZATION

(71) Applicant: GE Precision Healthcare LLC, Waukesha, WI (US)

(72) Inventors: Heng Zhao, Wauwatosa, WI (US); Brian Anthony Lause, Wauwatosa, WI (US)

(21) Appl. No.: 18/431,740

(22) Filed: Feb. 2, 2024

(51) Int. Cl. A61B 8/00; A61B 8/08

(52) U.S. Cl. CPC A61B 8/469; A61B 8/42; A61B 8/463; A61B 8/5207

(57) ABSTRACT

The current disclosure provides systems and methods for angle visualization in ultrasound imaging. In one example, a method includes obtaining ultrasound receive signals of a region of interest (ROI) of an imaging subject, generating a plurality of angle-emphasis images from the ultrasound receive signals, each angle-emphasis image generated from a weighted summation of the ultrasound receive signals, generating a spatial compound image from the plurality of angle-emphasis images, and displaying, on a display device, a plurality of overlaid images, each overlaid image including a respective angle-emphasis image of the plurality of angle-emphasis images overlaid on the spatial compound image.

### TECHNICAL FIELD

Embodiments of the subject matter disclosed herein relate generally to medical imaging, and more specifically, to visualization of ultrasound images and/or cine clips created from particular sets or subsets of insonification angles.

### BACKGROUND

Medical ultrasound is an imaging modality that employs ultrasound waves to probe the internal structures of a body of a patient and produce a corresponding image. For example, an ultrasound probe comprising a plurality of transducer elements emits ultrasonic pulses which reflect (e.g., echo), refract, or are absorbed by structures in the body. The ultrasound probe then receives reflected echoes, which are processed into an image. Ultrasound images of the internal structures may be saved for later analysis by a clinician to aid in diagnosis and/or the images may be displayed on a display device in real time or near real time.

### SUMMARY

In one example, a method includes obtaining ultrasound receive signals from a region of interest (ROI) of an imaging subject, generating a plurality of angle-emphasis images from the ultrasound receive signals, each angle-emphasis image generated from a different weighted summation of the ultrasound receive signals, generating a spatial compound image from the plurality of angle-emphasis images, and displaying, on a display device, a plurality of overlaid images, each overlaid image including a respective angle-emphasis image of the plurality of angle-emphasis images overlaid on the spatial compound image.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows a block diagram of an ultrasound system, according to an embodiment;

FIG. 2 shows a block schematic diagram of an image processing system, according to an embodiment;

FIG. 3 schematically shows an example process for generating a spatial compound image from ultrasound data obtained by the ultrasound system of FIG. 1 and performed by the image processing system of FIG. 2;

FIG. 4 schematically shows a plurality of example angle-emphasis images overlaid on the spatial compound image of FIG. 3;

FIG. 5 is a flowchart showing an example method for visualizing angle-emphasis and spatial compound images, according to an embodiment;

FIG. 6 schematically shows a first process for obtaining ultrasound data for generating angle-emphasis and spatial compound images, according to an embodiment;

FIG. 7 schematically shows a second process for obtaining ultrasound data for generating angle-emphasis and spatial compound images, according to an embodiment;

FIG. 8 schematically shows a third process for obtaining ultrasound data for generating angle-emphasis and spatial compound images, according to an embodiment;

FIG. 9 shows example angle-emphasis images used to form a spatial compound image; and

FIG. 10 shows example angle-emphasis images overlaid on the spatial compound image of FIG. 9.

### DETAILED DESCRIPTION

Systems and methods for visualizing angle-emphasis images and spatial compound images are provided herein. Due to the directivity of sound wave propagation, anisotropic structures (e.g., muscle fiber, vessel wall, or border of a lesion) may not be visualized as effectively as other structures when imaged with ultrasound imaging. Ultrasound imaging likewise may not effectively visualize structures behind a strong reflector (e.g., rib or calcification). One common method for combating this issue is spatial compounding, which typically includes transmission of ultrasound beams in multiple directions (e.g., multiple different transmit angles) and the compounding of the receive signals from these transmits into a single image. The compounding of the receive signals from the different transmits may include taking the mean, the maximum, or the minimum of the receive signals from the different transmits, or applying another method to suitably combine the receive signals. Spatial compounding has demonstrated that it not only improves border delineation and suppresses shadow, but also reduces noise and improves contrast resolution.

While a user may have several options to choose from for how to combine the receive signals, each method is typically fixed in that the contribution from each transmit angle to the final compounded image cannot be changed. However, the user may wish to see the structures from one or several specific angles to obtain more information, instead of looking at the final spatial compound image using a prescribed compounding method. For example, the user may gain more information regarding the shape or nature of a target such as a calcification or kidney stone, if the user can examine the reflection and the shadow of the target from multiple directions/transmit angles. Also, some anisotropic lesions may be isoechoic when scanned from some angles but become hypoechoic or hyperechoic when scanned from other angles. Because the spatial compounding method is fixed, the information from one angle may overshadow the information from another angle and the user may be unaware that a target is visualized differently at different angles. Further, the user may be unable to view one or more desired angles without adjusting the position of the ultrasound probe.

Thus, according to embodiments disclosed herein, a spatial compound image and the angle images (also referred to herein as angle-emphasis images) that make up the spatial compound image may be flexibility defined and displayed to allow a user more choice in which angle images are viewed. For example, a user may define a region of interest (ROI) and a plurality of angles to inspect the ROI from may be selected (either by the user or by the ultrasound imaging system). The selected angles may be dependent on the current transmit scan sequence, the location of the ROI relative to the ultrasound transducer, and the size of the ROI. For a given selected angle, the pre-spatial-compounding (coherent and incoherent) data within a range near the selected angle are isolated, formed into an angle-emphasis image, and overlaid on the original (compounded) image. The overlaying includes that some, but not all, of the overlaying image overlaps the underlying image. The user can adjust the weight (gain) of the selected angle-emphasis image to have a better view of the ROI under that angle. The user can repeat the above process for different angles (or the process may be carried out to generate multiple angle-emphasis images that are presented simultaneously), or the system can generate a cine clip (e.g., a video) showing the ROI inspected from each of the selected angles consecutively. The user can also choose a customized transmit scan sequence with specified steering/transmit angles, which generates a cine clip through additional image acquisition. Furthermore, the system could optimize a transmit scan sequence based on the location of the ROI and user specified angles to visualize the ROI from different angles more efficiently.

In some embodiments, the user may select a plurality of angles with high resolution (fine step size among angles, such as 1° rather than 5° between angles). And for each angle the system could, without transmit changes, modify the image reconstruction to emphasize all echoes in the image arising from the angle selected or as a window function about the selected angle. In this way, the user could quickly dial through a multitude of angles to reduce artifacts or optimize visibility of difficult to image structures. This could be applied especially in cases of muscle and nerve fibers, needle visualization, calcifications, stones, and hard to call lesions.

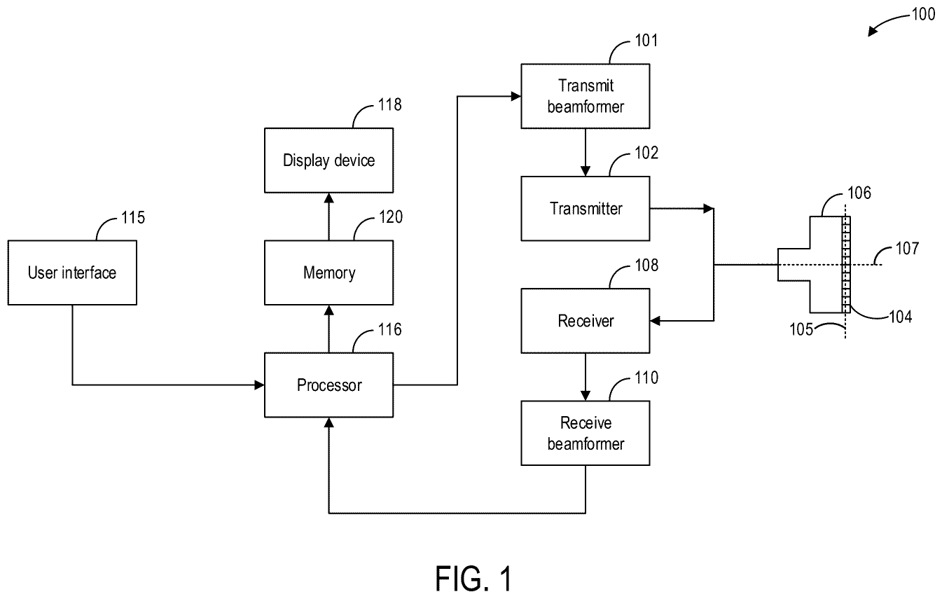

Referring to FIG. 1, a schematic diagram of an ultrasound imaging system 100 in accordance with an embodiment of the disclosure is shown. The ultrasound imaging system 100 includes a transmit beamformer 101 and a transmitter 102 that drives elements (e.g., transducer elements) 104 within a transducer array, herein referred to as probe 106, to emit pulsed ultrasonic signals (referred to herein as transmit pulses) into a body (not shown). According to an embodiment, the probe 106 may be a one-dimensional transducer array probe. However, in some embodiments, the probe 106 may be a two-dimensional matrix transducer array probe. As explained further below, the transducer elements 104 may be comprised of a piezoelectric material. When a voltage is applied to a piezoelectric crystal, the crystal physically expands and contracts, emitting an ultrasonic spherical wave. In this way, transducer elements 104 may convert electronic transmit signals into acoustic transmit beams.

After the elements 104 of the probe 106 emit pulsed ultrasonic signals into a body (of a patient), the pulsed ultrasonic signals are back-scattered from structures within an interior of the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs radio frequency (RF) data. Additionally, transducer element 104 may produce one or more ultrasonic pulses to form one or more transmit beams in accordance with the received echoes.

According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. A user interface 115 may be used to control operation of the ultrasound imaging system 100, including to control the input of patient data (e.g., patient medical history), to change a scanning or display parameter, to initiate a particular acquisition mode such as shear wave imaging, and the like. The user interface 115 may include one or more of the following: a rotary element, a mouse, a keyboard, a trackball, hard keys linked to specific actions, soft keys that may be configured to control different functions, and a graphical user interface displayed on a display device 118.

The ultrasound imaging system 100 also includes a processor 116 to control the transmit beamformer 101, the transmitter 102, the receiver 108, and the receive beamformer 110. The processor 116 is in electronic communication (e.g., communicatively connected) with the probe 106. For purposes of this disclosure, the term “electronic communication” may be defined to include both wired and wireless communications. The processor 116 may control the probe 106 to acquire data according to instructions stored on a memory of the processor, and/or memory 120. The processor 116 controls which of the elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processor (CPU), according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the RF data and generates IQ data pairs representative of the echo signals. In another embodiment, the demodulation can be carried out earlier in the processing chain. The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay. For example, an embodiment may acquire images at a real-time rate of 7-20 frames/sec. The ultrasound imaging system 100 may acquire 2D data of one or more planes at a significantly faster rate. However, it should be understood that the real-time frame-rate may be dependent on the length of time that it takes to acquire each frame of data for display. Accordingly, when acquiring a relatively large amount of data, the real-time frame-rate may be slower. Thus, some embodiments may have real-time frame-rates that are considerably faster than 20 frames/sec while other embodiments may have real-time frame-rates slower than 7 frames/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data, for example by augmenting the data, prior to displaying an image. It should be appreciated that other embodiments may use a different arrangement of processors.

The ultrasound imaging system 100 may continuously acquire data at a frame-rate of, for example, 10 Hz to 30 Hz (e.g., 10 to 30 frames per second). Images generated from the data may be refreshed at a similar frame-rate on display device 118. Other embodiments may acquire and display data at different rates. For example, some embodiments may acquire data at a frame-rate of less than 10 Hz or greater than 30 Hz depending on the size of the frame and the intended application. A memory 120 is included for storing processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

In various embodiments of the present invention, data may be processed in different mode-related modules by the processor 116 (e.g., B-mode, Color Doppler, M-mode, Color M-mode, spectral Doppler, Elastography, TVI, strain, strain rate, and the like) to form two-dimensional (2D) or three-dimensional (3D) data. For example, one or more modules may generate B-mode, color Doppler, M-mode, color M-mode, spectral Doppler, Elastography, TVI, strain, strain rate, and combinations thereof, and the like. As one example, the one or more modules may process color Doppler data, which may include traditional color flow Doppler, power Doppler, HD flow, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real time while a procedure (e.g., ultrasound imaging) is being performed on a patient. The video processor module may include a separate image memory, and the ultrasound images may be written to the image memory in order to be read and displayed by display device 118.

In various embodiments of the present disclosure, one or more components of ultrasound imaging system 100 may be included in a portable, handheld ultrasound imaging device. For example, display device 118 and user interface 115 may be integrated into an exterior surface of the handheld ultrasound imaging device, which may further contain processor 116 and memory 120. Probe 106 may comprise a handheld probe in electronic communication with the handheld ultrasound imaging device to collect raw ultrasound data. Transmit beamformer 101, transmitter 102, receiver 108, and receive beamformer 110 may be included in the same or different portions of the ultrasound imaging system 100. For example, transmit beamformer 101, transmitter 102, receiver 108, and receive beamformer 110 may be included in the handheld ultrasound imaging device, the probe, and combinations thereof.

After performing a two-dimensional ultrasound scan, a block of data comprising scan lines and their samples is generated. After back-end filters are applied, a process known as scan conversion is performed to transform the two-dimensional data block into a displayable bitmap image with additional scan information such as depths, angles of each scan line, and so on. During scan conversion, an interpolation technique is applied to fill missing holes (i.e., pixels) in the resulting image. These missing pixels occur because each element of the two-dimensional block should typically cover many pixels in the resulting image. For example, in current ultrasound imaging systems, a bicubic interpolation is applied which leverages neighboring elements of the two-dimensional block. As a result, if the two-dimensional block is relatively small in comparison to the size of the bitmap image, the scan-converted image will include areas of poor or low resolution, especially for areas of greater depth.

As used herein, an ultrasound transducer (or simply a transducer) may refer to a plurality of ultrasound transducer elements that are each capable of emitting and receiving ultrasound signals. In some examples, the ultrasound transducer may include each transducer element of the ultrasound probe. In some examples, the ultrasound transducer may be segmented into apertures, where each aperture includes at least one ultrasound transducer element. In spatial compound imaging, multiple transmits may be performed with the aperture(s) activated for transmitting and/or receiving ultrasound signals adjusted in order to insonify a target from multiple angles. In other examples, electronic beam steering (e.g., modified transmit focal positions) may be employed to insonify the target from multiple angles using the same aperture. The echoes resulting from a given transmit, whether received along the same angle as the transmit or from a different angle(s), may be processed to form an image, also referred to as an angle-emphasis image. The angle-emphasis images from the multiple transmits may be combined via weighted averaging or another suitable mechanism to form a spatial compound image.

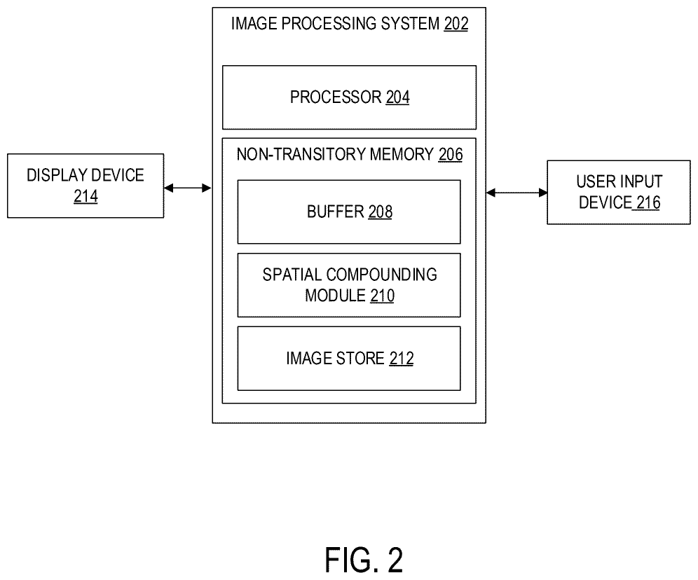

FIG. 2 is a block schematic diagram of an image processing system 202. In some embodiments, at least a portion of image processing system 202 is disposed at a device (e.g., workstation, edge device, server, etc.) communicably coupled to the ultrasound imaging system 100. In some examples, the image processing system 202 may be part of the ultrasound imaging system 100. For example, memory 120 and processor 116 may be incorporated in image processing system 202. Image processing system 202 may also be operably/communicatively coupled to a user input device 216 and a display device 214. As explained previously, the image processing system 202 may be part of the ultrasound imaging system 100, and thus the display device 214 may be the display device 118 of FIG. 1 and the user input device 216 may be the user interface 115 of FIG. 1.

Image processing system 202 includes a processor 204 configured to execute machine readable instructions stored in non-transitory memory 206. In some examples, processor 204 is a non-limiting example of processor 116. Processor 204 may be single core or multi-core, and the programs executed thereon may be configured for parallel or distributed processing. In some embodiments, processor 204 may optionally include individual components that are distributed throughout two or more devices, which may be remotely located and/or configured for coordinated processing. In some embodiments, one or more aspects of processor 204 may be virtualized and executed by remotely-accessible networked computing devices configured in a cloud computing configuration.

Non-transitory memory 206, which may be a non-limiting example of memory 120, may store a buffer 208, a spatial compounding module 210, and an image store 212. Buffer 208 may store recently-acquired ultrasound data (e.g., channel data, both coherent and incoherent, generated from receive signals output by the ultrasound transducer in response to ultrasound signals impinging on the transducer). Buffer 208 may be a first in-first out buffer that stores the previous 10 seconds, 30 seconds, or other suitable time/amount of ultrasound data acquired with the probe 106. Spatial compounding module 210 may include instructions that are executable by processor 204 to generate angle-emphasis images and spatial compound images, as well as select angles, transmit scan sequences, and the like, to facilitate the flexible angle selection and spatial compounding disclosed herein. Image store 212 may store ultrasound images and image loops (e.g., cine clips/videos) generated from the ultrasound data acquired with probe 106 (including angle-emphasis images and spatial compound images generated via spatial compounding module 210). As explained above with respect to FIG. 1, the ultrasound data acquired with the ultrasound probe may undergo a scan conversion process to form images. The images may be saved in image store 212. Image store 212 may be or include the image memory of the video processing module discussed above with respect to FIG. 1 and thus the images and image loops saved in image store 212 may be displayed on display device 214 in real-time during an exam. In some examples, additionally or alternatively, the images and image loops saved in image store 212 may be images and/or image loops that have been displayed and selected by a user for long-term storage.

User input device 216 may comprise one or more of a touchscreen, a keyboard, a mouse, a trackpad, a microphone, a motion sensing camera, or other device configured to enable a user to interact with image processing system 202. In one example, user input device 216 may enable a user to designate a ROI, select one or more angles for imaging, and so forth.

Display device 214 may include one or more display devices utilizing virtually any type of technology. In some embodiments, display device 214 may comprise a computer monitor. Display device 214 may be combined with processor 204, non-transitory memory 206, and/or user input device 216 in a shared enclosure, or may be peripheral display devices and may comprise a monitor, touchscreen, projector, or other display device known in the art, which may enable a user to view images and/or interact with various data stored in non-transitory memory 206.

It should be understood that image processing system 202 shown in FIG. 2 is for illustration, not for limitation. Another appropriate image processing system may include more, fewer, or different components.

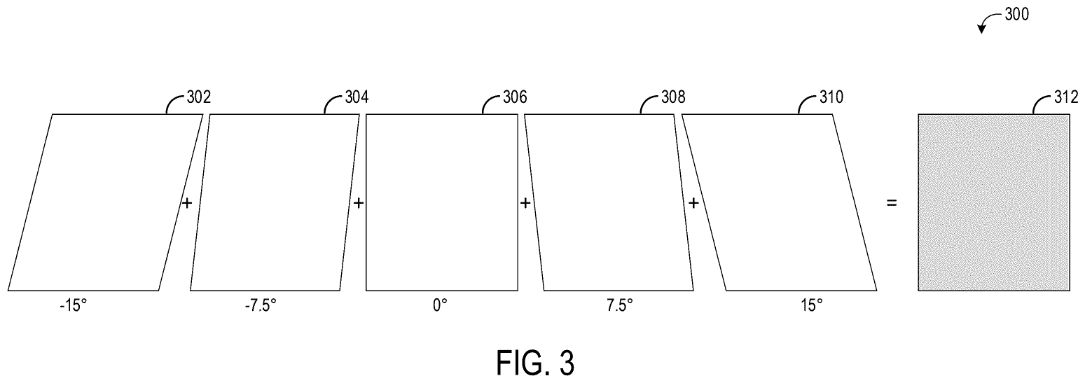

FIG. 3 schematically shows an example process 300 for generating a spatial compound image. As explained above, a target (e.g., an anatomical ROI such as an organ, muscle, lesion, needle, etc.) may be imaged from multiple angles to form component, angle-emphasis image frames. As shown, a first angle-emphasis image 302 may be generated from ultrasound signals (e.g., echoes) resulting from a first transmit performed at a first transmit angle. A second angle-emphasis image 304 may be generated from ultrasound signals resulting from a second transmit performed at a second transmit angle. A third angle-emphasis image 306 may be generated from ultrasound signals resulting from a third transmit performed at a third transmit angle. A fourth angle-emphasis image 308 may be generated from ultrasound signals resulting from a fourth transmit performed at a fourth transmit angle. A fifth angle-emphasis image 310 may be generated from ultrasound signals resulting from a fifth transmit performed at a fifth transmit angle.

In the example shown herein, the third transmit angle may be 0°, such that the transmitted ultrasound beam is directed perpendicular to a longitudinal axis of the transducer. For example, referring back to FIG. 1, the probe 106 includes transducer elements 104 that form the transducer of the probe 106. The transducer elements extend along a longitudinal axis 105. An ultrasound beam having an angle of 0° may extend perpendicular to the longitudinal axis 105 and parallel to the axis 107 shown in FIG. 1. The first transmit angle may be −15°, the second transmit angle may be −7.5°, the fourth transmit angle may be 7.5°, and the fifth transmit angle may be 15°, each of which may be relative to the axis 107. It is to be understood that the angles shown in FIG. 3 are exemplary and other angles may be used without departing from the scope of this disclosure. Further, as explained in more detail below, different angle-emphasis images may be generated by varying the receive angle instead of or in addition to varying the transmit angle. Similarly, more or fewer angle-emphasis image frames may be obtained and used to generate the spatial compound image, explained below.

The first angle-emphasis image 302, the second angle-emphasis image 304, the third angle-emphasis image 306, the fourth angle-emphasis image 308, and the fifth angle-emphasis image 310 may be combined to form a spatial compound image 312. For example, the first angle-emphasis image 302, the second angle-emphasis image 304, the third angle-emphasis image 306, the fourth angle-emphasis image 308, and the fifth angle-emphasis image 310 may be averaged over the area where all the images overlap to form the spatial compound image 312.

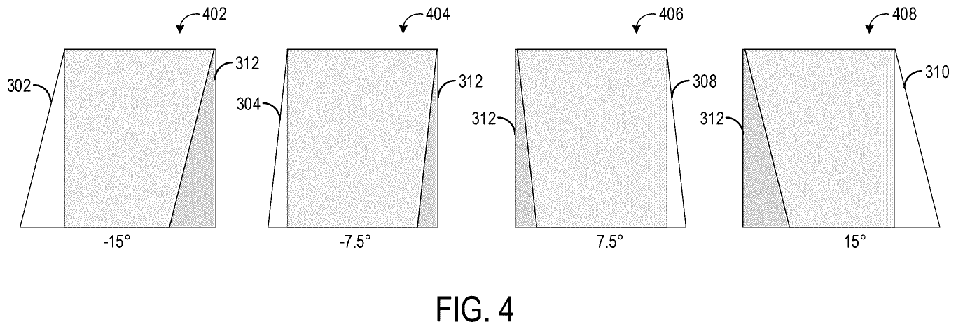

In conventional ultrasound imaging systems, the spatial compound image 312 may be displayed, e.g., on display device 214. However, as explained previously, relevant image information from one angle-emphasis image may be obscured or otherwise made less obvious to a user when the angle-emphasis images are combined into the spatial compound image. Thus, as shown schematically in FIG. 4, one or more or each of the angle-emphasis images may be displayed as an overlay on the spatial compound image. For example, as shown in FIG. 4, a first overlaid image 402 may include the spatial compound image 312 as a base image with the first angle-emphasis image 302 displayed on the spatial compound image 312 as an overlay, with the transparency and/or gain of each of the first angle-emphasis image 302 and the spatial compound image 312 set so that features in each of the first angle-emphasis image 302 and the spatial compound image 312 are visible. For example, the first angle-emphasis image 302 may have a transparency of greater than 0% and less than 100%, such as a transparency of 50%, while the spatial compound image 312 may have a transparency of 0%. In some examples, a user may be able to adjust the transparency and/or gain of the first angle-emphasis image 302 or both the first angle-emphasis image 302 and the spatial compound image 312 to set a desired contrast between the two images.

Similarly, a second overlaid image 404 may include the spatial compound image 312 as a base image with the second angle-emphasis image 304 displayed on the spatial compound image 312 as an overlay. A third overlaid image 406 may include the spatial compound image 312 as a base image with the fourth angle-emphasis image 308 displayed on the spatial compound image 312 as an overlay. A fourth overlaid image 408 may include the spatial compound image 312 as a base image with the fifth angle-emphasis image 310 displayed on the spatial compound image 312 as an overlay. Thus, by generating multiple different angle-emphasis images of a target, a user can view the target from different angles, which provides more information for anisotropic structures. The angle-emphasis images can be overlaid on top of the spatial compound image to provide some background context of the target.

In some examples, each of the overlaid images shown in FIG. 4 may be displayed simultaneously on a display device, such as display device 214, as shown in FIG. 4. In this way, the user may be able to compare the overlaid images to each other to determine which angle(s) provides desired information about the imaging target. In some examples, each overlaid image may be displayed sequentially at a suitable frame rate. For example, the overlaid images may be displayed as a video clip/image loop. The changes in the image frames from different angles may reveal more information about the shape and content of the anisotropic structure.

As will be explained in more detail below, in some examples, the user may request one or more specific angles or angle ranges at which the angle-emphasis images are to be generated and the ultrasound imaging system may acquire additional angle-emphasis images with specified transmit angles using a customized transmit scan sequence upon the user's request.

In some examples, different portions of the image could be constructed prioritizing different angularity, or each reconstruction location (roughly a pixel) could be constructed as a function of its own angularity. For example, each pixel of an ultrasound image may be formed by summing the corresponding signals from the receive elements (e.g., ultrasound transducer elements) based on the transmit and receive paths to and from each pixel. Specific angle(s) may be prioritized over other angles during summation (e.g., prioritizing may include assigning higher weights to the receive signals received at the specific angle(s) and/or resulting from transmits at the specific angle(s)). Therefore, different portions of the ultrasound image or even down to a pixel could have its own preferred/emphasized angularity. In this way, the reconstructed intensity of any location in the ultrasound image may be a weighted average of a plurality of intensities each from a different angle. This weighting function need not be shared across all image locations but can instead be different at every location in the image, or for different regions in the image. For example, when imaging a nerve bundle running roughly parallel to the skin line which then dives under a bone, a different weighting function, prioritizing different angles, could be used where the nerves run parallel as compared to where the nerves dive.

It is to be appreciated that while the term “angle-emphasis image” is used herein, each angle-emphasis image is not limited to ultrasound transmits angled at only one angle or ultrasound echoes received at only one angle. Rather, the angle-emphasis images described herein may include transmits angled at a range of angles (e.g.,) 5-10° centered around a specified angle and/or echoes received at a range of angles centered around a specified angle. Further, the ultrasound data obtained at the different angles may be obtained by varying the transmit angles, varying the receive angles, or both, as will be described in more detail below.

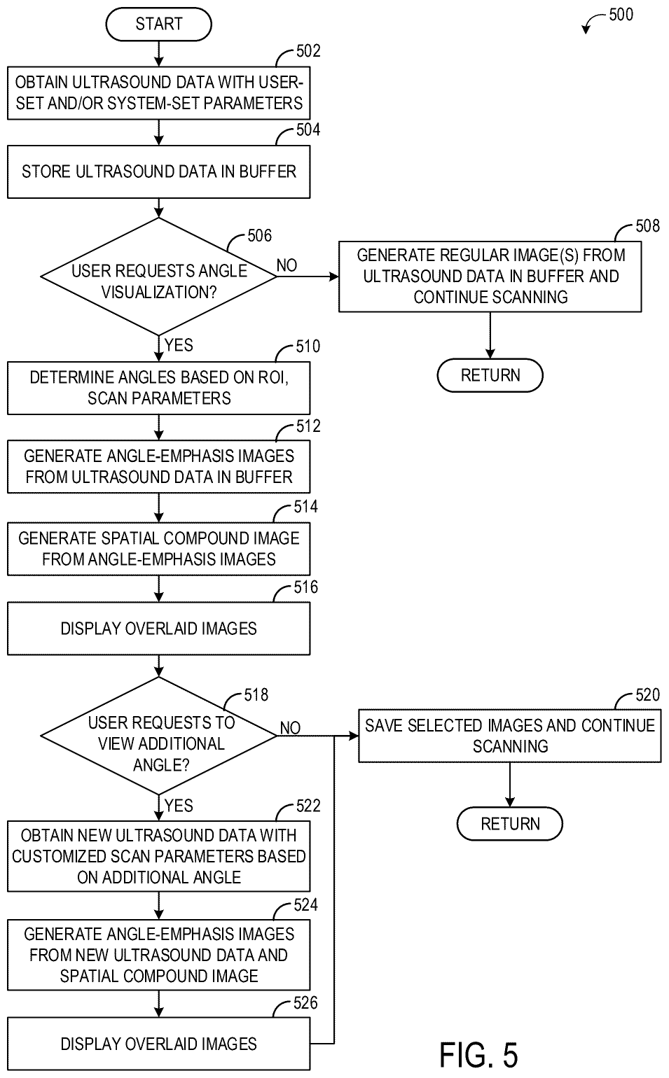

FIG. 5 shows a method 500 for spatial compound imaging according to embodiments of the disclosure. Method 500 may be implemented with the components of FIGS. 1 and 2. For example, method 500 may be performed by a processor of an ultrasound imaging system, such as processor 204 of image processing system 202 of FIG. 2, based on instructions stored in a non-transitory memory of the image processing system (e.g., stored as part of spatial compounding module 210).

At 502, method 500 includes obtaining ultrasound data with user-set and/or system-set parameters. The ultrasound data may be receive signals (e.g., channel data) after receive beamforming is performed, wherein the receive signals are obtained with an ultrasound probe and result from a transmit scan sequence having parameters set by a user (e.g., an operator of the ultrasound probe) and/or the ultrasound imaging system. The transmit scan sequence may dictate the shape, frequency, depth, angle, etc., of the transmitted ultrasound beams. The receive signals may be generated by the transducer of the ultrasound probe in response to echoes of the transmitted ultrasound beams received at the ultrasound probe, and the receive signals may also be based on user- and/or system-set parameters, including time delay, weighting, etc., applied to the receive signals. In some examples, the parameters used to obtain the ultrasound data may be based on the anatomy being imaged (e.g., the abdomen, the heart, etc.), the goal of the ultrasound exam (e.g., to diagnose a patient condition, track growth of a lesion, etc.), etc., which may be determined by the user and/or by the ultrasound system (e.g., based on a user-selected scan protocol).

At 504, the ultrasound data is stored in a buffer, such as buffer 208. The ultrasound data is usable to form one or more images. Further, the ultrasound data stored in the buffer may be data (e.g., receive signals/channel data) prior to undergoing scan conversion to form the one or more images. At 506, method 500 determines if the user has requested angle visualization, wherein a region of interest (ROI) that includes a target (e.g., a lesion) is visualized at multiple different transmit and/or receive angles, as both angle-emphasis images and a spatial compound image. The user may request angle visualization in a suitable manner, such as selecting a user interface element displayed on a display device of the ultrasound imaging system (e.g., a spatial compound button) or selecting a button on the ultrasound probe or on a keyboard of the ultrasound imaging system. If the user does not request angle visualization, method 500 proceeds to 508 to generate regular image(s) from the ultrasound data in the buffer and continue scanning. The regular image(s) may be from one angle, such that the receive signals used to form the regular image are generated from echoes of transmit beams at one angle and the echoes are received at the one angle, such as perpendicular to the transducer (e.g., the zero angle described above).

If the user has requested angle visualization, method 500 proceeds to 510 to determine the transmit and/or receive angles from which the angle-emphasis images will be generated based on the ROI and the scan parameters. For example, the distance between the transducer of the ultrasound probe and the ROI may dictate a range of possible transmit and/or receive angles, including a range of transmit angles at which the transmitted ultrasound beams may impinge the ROI and/or a range of receive angles at which the echoes from the ROI may be received at the transducer. The scan parameters, such as the aperture(s) used to transmit the ultrasound beams and/or the steering angle of each ultrasound beam, may also dictate the range of possible transmit and/or receive angles. Two or more angles may be selected from the range of possible transmit and/or receive angles. For example, if a range of 20° centered at 0° is the determined range of possible angles, the selected angles may be from within the range and may be separated by a suitable amount, such as by 5° (e.g., resulting in selected angles of 10°, 5°, 0°, −5°, and −10°). The angles may be selected automatically by the ultrasound imaging system (e.g., by the spatial compounding module 210) based on the range of possible angles and rules-based logic that dictates how many angles are to be selected and the separation between angles. In other examples, the angles may be selected by the user. The ROI may be identified by the user (e.g., via a user selection of depth for the scan and/or via user input to a previously-acquired image that is displayed on the display device).

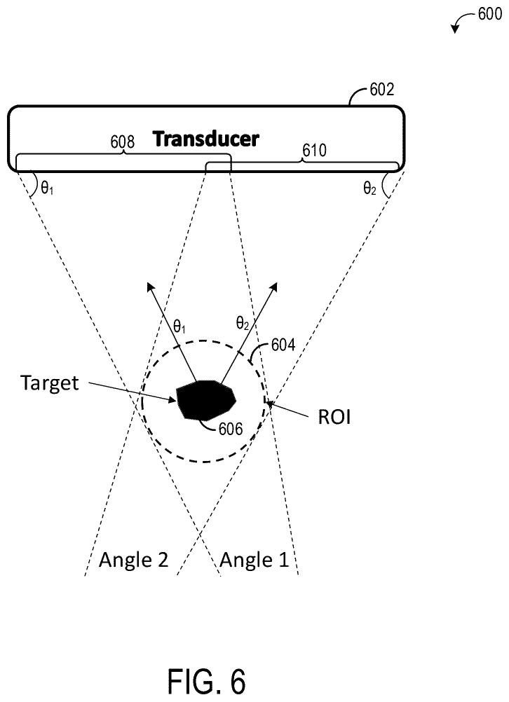

At 512, a plurality of angle-emphasis images is generated from the ultrasound data in the buffer, according to the angles selected at 510. Each angle-emphasis image may emphasize a different selected transmit and/or receive angle. For example, the transmit scan sequence performed to obtain the ultrasound data may include transmission of ultrasound beams at a plurality of different transmit angles. As a result, the echoes received from the transmitted ultrasound beams may generate ultrasound data originating from the different transmit angles. The ultrasound data may be processed into different subsets of ultrasound data based on the transmit angles selected at 510, such that each subset of the ultrasound data includes ultrasound data generated by the ultrasound probe from echoes of ultrasound beams transmitted at a respective selected transmit angle of the plurality of different transmit angles. As used herein, a subset of set X means a portion, but less than all, of set X. In other examples, the receive angle may be adjusted with a constant transmit angle, or both the receive angle and the transmit angle may be adjusted, as shown in FIGS. 6-8 and explained below. The processing of the ultrasound data to form the plurality of angle-emphasis images may include performing different weighted summations of the receive signals (e.g., the ultrasound data) to emphasize the selected angles. For example, to generate a first angle-emphasis image emphasizing echoes resulting from transmits at and/or in a range around a first angle (or echoes received at the ultrasound transducer at or in a range around the first angle), a first weighted summation of the receive signals may be performed so that the first angle-emphasis image is reconstructed from only the receive signals at and/or in a range around the first angle. To generate a second angle-emphasis image emphasizing echoes resulting from transmits at and/or in a range around a second angle (or echoes received at the ultrasound transducer at or in a range around the second angle), a second weighted summation of the receive signals may be performed so that the second angle-emphasis image is reconstructed from only the receive signals at and/or in a range around the second angle. It is to be appreciated that in some examples, a given angle-emphasis image may be reconstructed from receive signals at and/or in a range around the specified angle and receive signals of echoes at other angles, with the receive signals at and/or in a range around the specified angle weighted more than the other angles to emphasize the specified angle.

For example, FIG. 6 schematically shows a scan sequence 600 that includes two different transmit angles. The scan sequence may be performed with an ultrasound probe including a transducer 602 comprising a plurality of ultrasound transducer elements, as explained above with respect to FIG. 1. The transducer 602 may be controlled during the scan sequence to transmit ultrasound beams to an ROI 604 of an imaging subject (e.g., a patient) that includes a target 606 (e.g., a lesion). The ultrasound beams may be transmitted at a plurality of transmit angles including a first transmit angle and a second transmit angle, as shown by the dotted lines in FIG. 6. The ultrasound beams transmitted at the first transmit angle (01) may be transmitted from a first aperture 608 of the transducer 602 that includes a first subset of the transducer elements. Beam steering may be performed to direct the ultrasound beams at the first transmit angle. The ultrasound beams transmitted at the second transmit angle (02) may be transmitted from a second aperture 610 of the transducer 602 that includes a second subset of the transducer elements, with beam steering performed to direct the ultrasound beams at the second transmit angle. The echoes received at the transducer 602 from the ultrasound beams transmitted at the first transmit angle and received at the same angle (e.g., as shown by the solid line labeled θ₁) or in a range of angles around the same angle (e.g., within 1-5° of the transmit angle) may be processed to form a first subset of ultrasound data (e.g., a first subset of receive signals). Likewise, the echoes received at the transducer 602 from the ultrasound beams transmitted at the second transmit angle and received at the same angle (e.g., as shown by the solid line labeled θ₂) or in a range of angles around the same angle (e.g., within 1-5° of the transmit angle) may be processed to form a second subset of ultrasound data (e.g., a second subset of receive signals). A first angle-emphasis image may be generated from the first subset of ultrasound data and a second angle-emphasis image may be generated from the second subset of ultrasound data.

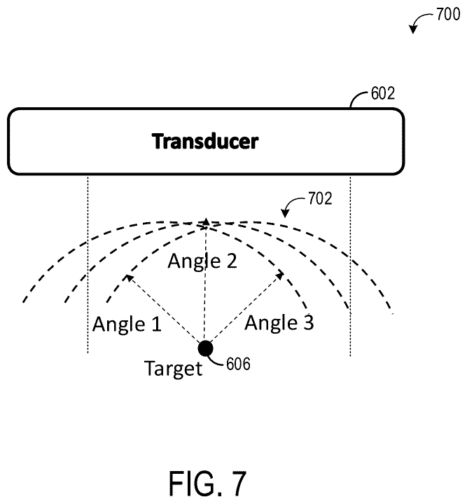

However, other mechanisms are possible for generating the angle-emphasis images, such as by varying the receive angle rather than (or in addition to) the transmit angle. FIG. 7 schematically shows a scan sequence 700 that includes three different receive angles. The scan sequence may be performed with an ultrasound probe including the transducer 602 comprising a plurality of ultrasound transducer elements, as explained above with respect to FIG. 1. As explained above with respect to FIG. 6, the transducer 602 may be controlled to transmit ultrasound beams to an ROI of an imaging subject (e.g., a patient) that includes the target 606. The ultrasound beams may be transmitted at one transmit angle in the example shown in FIG. 7 (e.g., the zero angle), as shown by the dotted lines extending from the transducer 602. Each transmit may result in an echo(es) 702 from the target 606 that is eventually received at the transducer 602. The transducer elements of the transducer 602 may generate receive signals in response to the echo(es) that may be processed to form subsets of ultrasound data/receive signals associated with different receive angles. For example, three receive angles are shown in FIG. 7, as shown by the dotted lines and labeled Angle 1, Angle 2, and Angle 3. The receive signals may be determined to be associated with a given receive angle (and hence processed into a given subset of ultrasound data) based on the time since the transmit that the echo(es) was received at the transducer 602 (e.g., echoes from the second receive angle may arrive at the transducer 602 sooner than echoes from the first receive angle and the third receive angle) and the transducer elements that receive the echo(es).

The echo(es) received at the transducer 602 at the first receive angle (or within a range of the first receive angle) may be processed to form a first subset of ultrasound data (e.g., a first subset of receive signals), the echo(es) received at the transducer 602 at the second receive angle (or within a range of the second receive angle) may be processed to form a second subset of ultrasound data (e.g., a second subset of receive signals), and the echo(es) received at the transducer 602 at the third receive angle (or within a range of the third receive angle) may be processed to form a third subset of ultrasound data (e.g., a third subset of receive signals). In some examples, the processing may include performing a first weighted summation of the receive signals resulting from the echoes received at the transducer from all the angles to emphasize the first angle (e.g., receive signals representing echoes received at the second and third angles may be weighted 0), performing a second weighted summation of the receive signals resulting from the echoes received at the transducer from all the angles to emphasize the second angle, and performing a third weighted summation of the receive signals resulting from the echoes received at the transducer from all the angles to emphasize the third angle. A first angle-emphasis image may be generated from the first subset of ultrasound data (e.g., from the first weighted summation), a second angle-emphasis image may be generated from the second subset of ultrasound data (e.g., from the second weighted summation), and a third angle-emphasis image may be generated from the third subset of ultrasound data (e.g., from the third weighted summation).

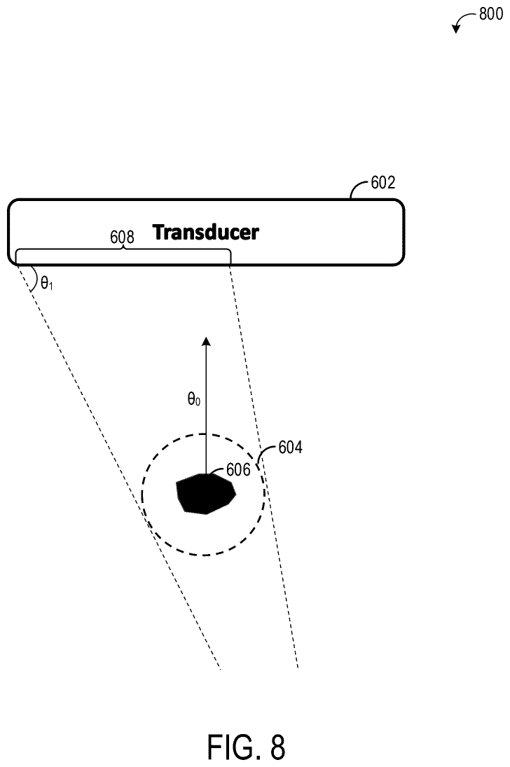

FIG. 8 schematically shows another example scan sequence 800 that may be performed with an ultrasound probe including the transducer 602 that is controlled to transmit ultrasound beams to the ROI 604 of an imaging subject (e.g., a patient) that includes the target 606 (e.g., a lesion). The ultrasound beams may be transmitted at one or more transmit angles including the first transmit angle via the first aperture 608, as explained above with respect to FIG. 6. Each transmit at the first transmit angle may result in an echo(es) from the target 606 that is eventually received at the transducer 602. The transducer elements of the transducer 602 may generate receive signals in response to the echo(es) that may be processed to form one or more subsets of ultrasound data/receive signals associated with one or more receive angles that may be different than the transmit angle. In the example shown in FIG. 8, the receive signals that are processed to form a first subset of ultrasound data may be received at a different angle or range of angles than the transmit angle, such as the zero angle as shown in FIG. 8 (e.g., θ₀). As explained previously, the first subset of ultrasound data may be used to generate a first angle-emphasis image, and additional angle-emphasis images may be generated by adjusting the receive angle and/or the transmit angle.

Thus, FIGS. 6-8 show that angle-emphasis images may be generated by varying the transmit angle with the receive angle matching the transmit angle (as shown in FIG. 6), varying the receive angle with a single transmit angle (as shown in FIG. 7), or varying the transmit angle and/or the receive angle with the receive angle not matching the transmit angle (as shown in FIG. 8).

Returning to FIG. 5, at 514, method 500 includes generating a spatial compound image from the angle-emphasis images. The spatial compound image may be generated by averaging the angle-emphasis images or combining the angle-emphasis images according to another suitable method. At 516, overlaid images are displayed. Each angle-emphasis image may be overlaid on the spatial compound image and displayed as a respective overlaid image. For example, if three angles are selected and thus three angle-emphasis images are generated, a first overlaid image may be generated that includes a first angle-emphasis image overlaid on the spatial compound image, a second overlaid image may be generated that includes a second angle-emphasis image overlaid on the spatial compound image, and a third overlaid image may be generated that includes a third angle-emphasis image overlaid on the spatial compound image. The overlaid images may be displayed simultaneously and/or sequentially (e.g., as a video). In some examples, the spatial compound image may also be displayed. As explained previously, some features may be visualized differently depending on the angle at which the feature is insonified. Thus, by generating and displaying overlaid images with different angle-emphasis images, these features may be made more apparent to a user. For example, the first overlaid image may show a feature as being isoechoic while the third overlaid image may show the feature as being hyperechoic. By displaying multiple overlaid images generated from different angle-emphasis images, the user may be able to more readily identify the feature relative to only displaying the spatial compound image. Further, a feature that changes in position as a function of the insonification angle may be identified as a shadow of a reflective surface, instead of a hypoechoic region with clinical implications.

At 518, method 500 determines if the user has requested to view additional angles not included in the angle-emphasis images generated at 512. As explained previously, the transmit scan sequence performed to generate the initial ultrasound data may be limited in the angle-emphasis images that can be generated from the initial ultrasound data. Thus, the user may decide after viewing the overlaid images that the target should be viewed from additional angles and enter user input requesting more angles. The user may request specific angle(s) or a range of angles to view (e.g., the user may request to view three additional angles that are smaller or larger than the already-viewed angles).

If the user does not request to view additional angles, method 500 proceeds to 520 to save any selected images and continue scanning. For example, when the user views the overlaid images displayed at 516, the user may enter user input selecting one or more of the overlaid images and/or the spatial compound image for long-term storage (e.g., as part of a patient exam). If the user does request to view additional angles, method 500 proceeds to 522 to obtain new ultrasound data with customized scan parameters based on the additional angles. For example, new transmit scan parameters may be selected such that ultrasound beams may be transmitted at the additional angles. As a non-limiting example, if the angles imaged previously (e.g., to generate the angle-emphasis images at 512) were in a range of 10° to −10°, the user may request to view the target at 20°, 15°,−15°, and −20°, and a new transmit scan sequence may be set and performed to steer the ultrasound beams to 20°, 15°,−15°, and −20° in order to obtain the new ultrasound data.

At 524, new angle-emphasis images are generated from the new ultrasound data, according to the angles selected by the user. Each angle-emphasis image may correspond to a different selected transmit and/or receive angle. Further, the new angle-emphasis images may be combined to form a new spatial compound image. At 526, new overlaid images are displayed. Each new angle-emphasis image may be overlaid on the new spatial compound image and displayed as a respective overlaid image. At 520, any selected images may be saved, as described previously, and scanning may continue or end if the user determines all desired images have been acquired.

Thus, method 500 includes transmitting a plurality of ultrasound waves at varying angles to a target/ROI (e.g., according to the transmit scan sequence as described above), collecting echoes from the transmitted plurality of ultrasound waves (e.g., generating ultrasound receive signals via the transducer in response to the echoes impinging on the transducer), reconstructing an image prioritizing echoes at or near a specific angle (e.g., generating an angle-emphasis image from the receive signals as described above), and displaying the image. The image may be overlaid on a spatial compound image, as explained above.

With a standard (e.g., generic) transmit scan sequence, the user may only be able to view limited angle-emphasis images based on the available angles that can be calculated using the existing ultrasound data collected from the transmit scan sequence. Therefore, the user has to choose from available angle options. If the user wants to view specific angles not supported by the generic transmit scan sequence, then the ultrasound imaging system may generate a customized transmit scan sequence to image at the specified angles, which demands additional image acquisition (transmit and receive). As used herein, “generic” means the typical scan/transmit configuration used for regular ultrasound imaging. The “customized” or “optimized” scan configuration is generated based on user's request for additional viewing angles.

Further, while FIG. 5 was explained above as generating/reconstructing images with the same angle emphasized across an entirety of a given image, different regions (e.g., pixels or pixel groups) of an angle-emphasis image could emphasize different angles. Further, rather than the user specifying the additional angles, the ultrasound imaging system may be configured to analyze the initial angle-emphasis images and/or spatial compound image and automatically select additional angles at which the additional angle-emphasis images are to emphasize.

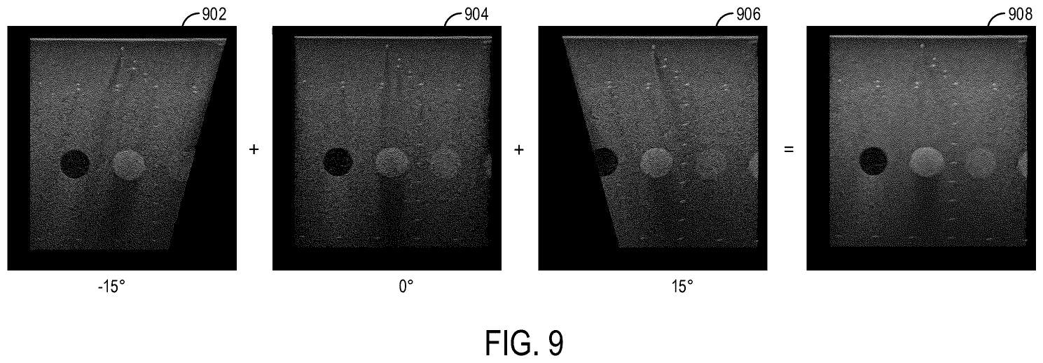

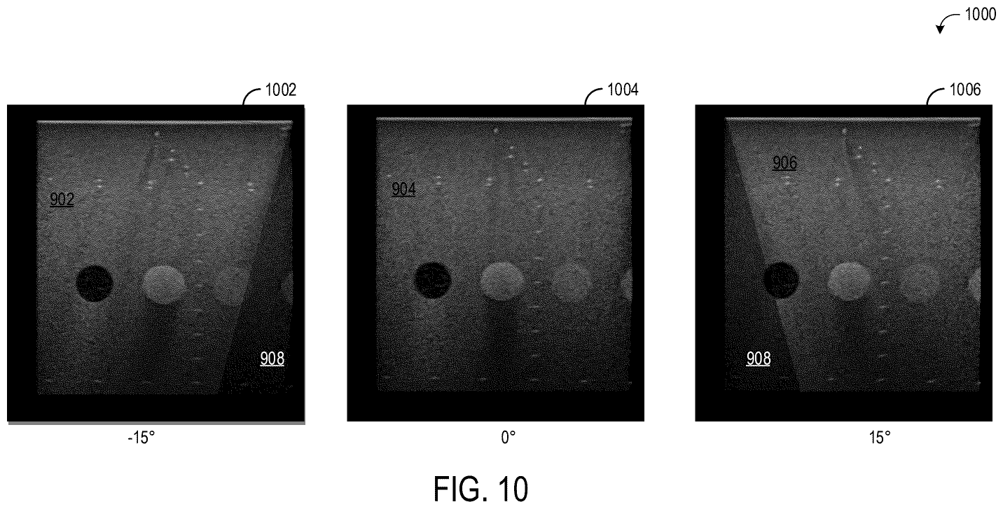

FIGS. 9 and 10 show example images of a phantom generated according to embodiments of the disclosure. FIG. 9 shows a plurality of angle-emphasis images combined to form a spatial compound image. Specifically, a first angle-emphasis image 902, a second angle-emphasis image 904, and a third angle-emphasis image 906 may be combined to form a spatial compound image 908. The first angle-emphasis image 902 may be generated from ultrasound data obtained as a result of ultrasound beams transmitted at a first angle of −15°, the second angle-emphasis image 904 may be generated from ultrasound data obtained as a result of ultrasound beams transmitted at a second angle of 0°, and the third angle-emphasis image 906 may be generated from ultrasound data obtained as a result of ultrasound beams transmitted at a third angle of 15°, though it is to be appreciated that the angles are exemplary and non-limiting, and that the angle-emphasis images could be generated by varying the receive angle instead of or in addition to the transmit angle.

FIG. 10 shows a plurality of overlaid images 1000 that may be generated as described herein. The plurality of overlaid images 1000 may include a first overlaid image 1002, a second overlaid image 1004, and a third overlaid image 1006. The first overlaid image 1002 may include the spatial compound image 908 and the first angle-emphasis image 902 overlaid on the spatial compound image 908. The second overlaid image 1004 may include the spatial compound image 908 and the second angle-emphasis image 904 overlaid on the spatial compound image 908. The third overlaid image 1006 may include the spatial compound image 908 and the third angle-emphasis image 906 overlaid on the spatial compound image 908. The plurality of overlaid images 1000 may be displayed on a display device (e.g., display device 214) as separate images, simultaneously, as shown. In other examples, the plurality of overlaid images 1000 may be displayed sequentially, such as a video/image loop at a suitable frame rate. As appreciated from FIGS. 9 and 10, a dark region present in the angle-emphasis images may shift as a function of the insonification angle. For example, the dark region in the first overlaid image 1002 may extend at approximately −15° while the dark region in the third overlaid image 1006 may extend at approximately 15°. The shifting angle may suggest the dark region is a shadow caused by a reflective surface (e.g., calcification) instead of a hyperechoic region underneath that may indicate lesions or other structures. The shifting of the dark region may not be apparent when viewing only the spatial compound image.

Thus, the systems and methods described herein allow for flexible definition and display of different angle images. Currently, users can select a single angle (B steer) at which a target may be visualized, or users can select spatial compounding methods that combine data from different angles following a fixed algorithm. However, data from other angles may overshadow the angle that reveals the important information of the target. The user can turn off spatial compounding and manually manipulate the probe to scan in different angles. However, it is easier for a user to obtain information from a relatively stationary target inspected at different angles, rather than from a constantly moving target. Accordingly, by displaying the angle-emphasis images separately (as the overlaid images), the user may determine an angle at which a target is optimally visualized. The user can either activate this feature using the acquired data with preferred configurations (e.g., default scan parameters with selected/limited angles) or choose to have more available angles with an additional image acquisition, without having to move the ultrasound probe.

A technical effect of generating and displaying a plurality of overlaid images, each overlaid image including a respective angle-emphasis image overlaid on a spatial compound image, is certain anatomical features may be better visualized at different angles and the overlaid images allow a user to visualize the separate angle-emphasis images relative to the spatial compound image so that background context can be visualized.

In another representation, a method for an image processing system of an ultrasound imaging system includes performing a first transmit scan sequence with an ultrasound probe of the ultrasound imaging system to transmit a first plurality of ultrasound beams at least at a first transmit angle and a second transmit angle to a region of interest (ROI) of an imaging subject; obtaining first ultrasound receive signals of the ROI from the ultrasound probe in response to the first transmit scan sequence; generating a plurality of first overlaid images from the first ultrasound receive signals, each first overlaid image including a respective first angle-emphasis image of a plurality of first angle-emphasis images overlaid on a first spatial compound image, each first angle-emphasis image of the plurality of first angle-emphasis images generated from the first ultrasound receive signals, the first spatial compound image generated from the plurality of first angle-emphasis images; receiving a user input requesting additional transmit angles, and in response, performing a second transmit scan sequence with the ultrasound probe to transmit a second plurality of ultrasound beams at least at a third transmit angle to the ROI; obtaining second ultrasound receive signals of the ROI from the ultrasound probe in response to the second transmit scan sequence; generating a plurality of second overlaid images from the second ultrasound receive signals, each second overlaid image including a respective second angle-emphasis image of a plurality of second angle-emphasis images overlaid on a second spatial compound image, each second angle-emphasis image of the plurality of second angle-emphasis images generated from the second ultrasound receive signals, the second spatial compound image generated from the plurality of second angle-emphasis images; and displaying the plurality of second overlaid images on a display device. In a first example of the method, each first angle-emphasis image corresponds to a different transmit angle of a plurality of transmit angles of the first transmit scan sequence, the plurality of transmit angles including the first transmit angle and the second transmit angle, and wherein the plurality of transmit angles is selected based on a size of the ROI and a location of the ROI relative to the ultrasound probe. In a second example of the method, optionally including the first example, the third transmit angle is selected based on the user input.

The disclosure also provides support for a method, comprising: obtaining ultrasound receive signals from a region of interest (ROI) of an imaging subject, generating a plurality of angle-emphasis images from the ultrasound receive signals, each angle-emphasis image generated from a different weighted summation of the ultrasound receive signals, generating a spatial compound image from the plurality of angle-emphasis images, and displaying, on a display device, a plurality of overlaid images, each overlaid image including a respective angle-emphasis image of the plurality of angle-emphasis images overlaid on the spatial compound image. In a first example of the method, the method further comprises: performing a transmit scan sequence with an ultrasound probe to transmit a plurality of ultrasound beams to the ROI, and wherein the ultrasound receive signals are collected by the ultrasound probe from echoes of the plurality of ultrasound beams. In a second example of the method, optionally including the first example, performing the transmit scan sequence comprises transmitting the plurality of ultrasound beams at a plurality of different transmit angles, and wherein a respective ultrasound receive signal dataset is collected by the ultrasound probe from echoes of each ultrasound beam of the plurality of ultrasound beams transmitted at a respective angle of the plurality of different transmit angles, and wherein the plurality of angle-emphasis images is generated from one or more of the ultrasound receive signal datasets. In a third example of the method, optionally including one or both of the first and second examples, the ultrasound receive signals includes one or more ultrasound receive signal datasets each collected by the ultrasound probe from echoes received at a respective angle of a plurality of different receive angles. In a fourth example of the method, optionally including one or more or each of the first through third examples, each angle-emphasis image corresponds to a different transmit and/or receive angle of a plurality of transmit and/or receive angles, and wherein the plurality of transmit and/or receive angles are selected in response to user input. In a fifth example of the method, optionally including one or more or each of the first through fourth examples, the transmit scan sequence includes one or more parameters selected based on a size of the ROI and the plurality of transmit and/or receive angles. In a sixth example of the method, optionally including one or more or each of the first through fifth examples, each angle-emphasis image corresponds to a different transmit and/or receive angle of a plurality of transmit and/or receive angles, and wherein the plurality of transmit and/or receive angles are selected based on a size of the ROI and a location of the ROI relative to the ultrasound probe. In a seventh example of the method, optionally including one or more or each of the first through sixth examples, displaying, on the display device, the plurality of overlaid images comprises displaying the plurality of overlaid images in sequence as a video.

The disclosure also provides support for an image processing system, comprising a processor and a non-transitory memory storing instructions that when executed, cause the processor to: obtain ultrasound receive signals of a region of interest (ROI) of an imaging subject, generate a plurality of angle-emphasis images from the ultrasound receive signals, each angle-emphasis image generated from a respective subset of the ultrasound receive signals, generate a spatial compound image from the plurality of angle-emphasis images, and display, on a display device, a plurality of overlaid images, each overlaid image including a respective angle-emphasis image of the plurality of angle-emphasis images overlaid on the spatial compound image. In a first example of the system, the instructions, when executed, further cause the processor to perform a transmit scan sequence with an ultrasound probe to transmit a plurality of ultrasound beams to the ROI, and wherein the ultrasound receive signals are generated by the ultrasound probe from echoes of the plurality of ultrasound beams. In a second example of the system, optionally including the first example, performing the transmit scan sequence comprises transmitting the plurality of ultrasound beams at a plurality of different transmit angles, and wherein each subset of the ultrasound receive signals is generated by the ultrasound probe from echoes of one or more ultrasound beams of the plurality of ultrasound beams transmitted at a respective angle of the plurality of different transmit angles. In a third example of the system, optionally including one or both of the first and second examples, each angle-emphasis image corresponds to a different weighted summation of transmit and/or receive angles of a plurality of transmit and/or receive angles, and wherein the plurality of transmit and/or receive angles are selected in response to user input. In a fourth example of the system, optionally including one or more or each of the first through third examples, the transmit scan sequence includes one or more parameters selected based on a size of the ROI and the plurality of transmit and/or receive angles. In a fifth example of the system, optionally including one or more or each of the first through fourth examples, each angle-emphasis image corresponds to a different transmit and/or receive angle of a plurality of transmit and/or receive angles, and wherein the plurality of transmit and/or receive angles are selected based on a size of the ROI and a location of the ROI relative to the ultrasound probe. In a sixth example of the system, optionally including one or more or each of the first through fifth examples, displaying, on the display device, the plurality of overlaid images comprises displaying the plurality of overlaid images in sequence as a video.

The disclosure also provides support for a method for generation of an ultrasound image, comprising: transmitting a plurality of ultrasound waves at varying angles to a target, collecting echoes from the transmitted plurality of ultrasound waves, reconstructing an image prioritizing echoes at or near a specific angle, and displaying the image. In a first example of the method, the specific angle is selected based on user input and wherein reconstructing the image comprises reconstructing the image prioritizing echoes at or near the specific angle across an entirety of the image. In a second example of the method, optionally including the first example, the specific angle is selected based on user input and wherein reconstructing the image comprises reconstructing the image prioritizing echoes at or near the specific at only a selected region of the image and prioritizing echoes at or near one or more different angles for a remainder of the image. In a third example of the method, optionally including one or both of the first and second examples, the specific angle is selected automatically and wherein reconstructing the image comprises reconstructing the image prioritizing echoes at or near the specific angle across an entirety of the image. In a fourth example of the method, optionally including one or more or each of the first through third examples, the specific angle is selected automatically and wherein reconstructing the image comprises reconstructing the image prioritizing echoes at or near the specific at only a selected region of the image and prioritizing echoes at or near one or more different angles for a remainder of the image.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative and should not be construed to be limiting in any manner.

1. A method, comprising:
obtaining ultrasound receive signals from a region of interest (ROI) of an imaging subject;
generating a plurality of angle-emphasis images from the ultrasound receive signals, each angle-emphasis image generated from a different weighted summation of the ultrasound receive signals;
generating a spatial compound image from the plurality of angle-emphasis images; and
displaying, on a display device, a plurality of overlaid images, each overlaid image including a respective angle-emphasis image of the plurality of angle-emphasis images overlaid on the spatial compound image.

2. The method of claim 1, further comprising performing a transmit scan sequence with an ultrasound probe to transmit a plurality of ultrasound beams to the ROI, and wherein the ultrasound receive signals are collected by the ultrasound probe from echoes of the plurality of ultrasound beams.

3. The method of claim 2, wherein performing the transmit scan sequence comprises transmitting the plurality of ultrasound beams at a plurality of different transmit angles, and wherein a respective ultrasound receive signal dataset is collected by the ultrasound probe from echoes of each ultrasound beam of the plurality of ultrasound beams transmitted at a respective angle of the plurality of different transmit angles, and wherein the plurality of angle-emphasis images is generated from one or more of the ultrasound receive signal datasets.

4. The method of claim 2, wherein the ultrasound receive signals includes one or more ultrasound receive signal datasets each collected by the ultrasound probe from echoes received at a respective angle of a plurality of different receive angles.

5. The method of claim 2, wherein each angle-emphasis image corresponds to a different transmit and/or receive angle of a plurality of transmit and/or receive angles, and wherein the plurality of transmit and/or receive angles are selected in response to user input.

6. The method of claim 5, wherein the transmit scan sequence includes one or more parameters selected based on a size of the ROI and the plurality of transmit and/or receive angles.

7. The method of claim 2, wherein each angle-emphasis image corresponds to a different transmit and/or receive angle of a plurality of transmit and/or receive angles, and wherein the plurality of transmit and/or receive angles are selected based on a size of the ROI and a location of the ROI relative to the ultrasound probe.

8. The method of claim 1, wherein displaying, on the display device, the plurality of overlaid images comprises displaying the plurality of overlaid images in sequence as a video.

9. An image processing system, comprising a processor and a non-transitory memory storing instructions that when executed, cause the processor to:
obtain ultrasound receive signals of a region of interest (ROI) of an imaging subject;
generate a plurality of angle-emphasis images from the ultrasound receive signals, each angle-emphasis image generated from a respective subset of the ultrasound receive signals;
generate a spatial compound image from the plurality of angle-emphasis images; and
display, on a display device, a plurality of overlaid images, each overlaid image including a respective angle-emphasis image of the plurality of angle-emphasis images overlaid on the spatial compound image.

10. The system of claim 9, wherein the instructions, when executed, further cause the processor to perform a transmit scan sequence with an ultrasound probe to transmit a plurality of ultrasound beams to the ROI, and wherein the ultrasound receive signals are generated by the ultrasound probe from echoes of the plurality of ultrasound beams.

11. The system of claim 10, wherein performing the transmit scan sequence comprises transmitting the plurality of ultrasound beams at a plurality of different transmit angles, and wherein each subset of the ultrasound receive signals is generated by the ultrasound probe from echoes of one or more ultrasound beams of the plurality of ultrasound beams transmitted at a respective angle of the plurality of different transmit angles.

12. The system of claim 10, wherein each angle-emphasis image corresponds to a different weighted summation of transmit and/or receive angles of a plurality of transmit and/or receive angles, and wherein the plurality of transmit and/or receive angles are selected in response to user input.

13. The system of claim 12, wherein the transmit scan sequence includes one or more parameters selected based on a size of the ROI and the plurality of transmit and/or receive angles.

14. The system of claim 10, wherein each angle-emphasis image corresponds to a different transmit and/or receive angle of a plurality of transmit and/or receive angles, and wherein the plurality of transmit and/or receive angles are selected based on a size of the ROI and a location of the ROI relative to the ultrasound probe.

15. The system of claim 9, wherein displaying, on the display device, the plurality of overlaid images comprises displaying the plurality of overlaid images in sequence as a video.

16. A method for generation of an ultrasound image, comprising:
transmitting a plurality of ultrasound waves at varying angles to a target;
collecting echoes from the transmitted plurality of ultrasound waves;
reconstructing an image prioritizing echoes at or near a specific angle; and
displaying the image.

17. The method of claim 16, wherein the specific angle is selected based on user input and wherein reconstructing the image comprises reconstructing the image prioritizing echoes at or near the specific angle across an entirety of the image.

18. The method of claim 16, wherein the specific angle is selected based on user input and wherein reconstructing the image comprises reconstructing the image prioritizing echoes at or near the specific at only a selected region of the image and prioritizing echoes at or near one or more different angles for a remainder of the image.

19. The method of claim 16, wherein the specific angle is selected automatically and wherein reconstructing the image comprises reconstructing the image prioritizing echoes at or near the specific angle across an entirety of the image.

20. The method of claim 16, wherein the specific angle is selected automatically and wherein reconstructing the image comprises reconstructing the image prioritizing echoes at or near the specific at only a selected region of the image and prioritizing echoes at or near one or more different angles for a remainder of the image.
